(19) United States
(12) Patent Application Publication
(10) Pub. No.: US 2022/0184265 A1
(43) Pub. Date: Jun. 16, 2022

(54) Antibacterial Spray Release Apparatus

(71) Applicant: Ashley Livingston, Battleground, WA (US)

(72) Inventor: Ashley Livingston, Battleground, WA (US)

(21) Appl. No.: 17/124,026

(22) Filed: Dec. 16, 2020

(51) Int. Cl. A61L 9/14

(52) U.S. Cl. CPC A61L 9/14; A61L 2209/111; A61L 2209/134; A61L 2209/133

(57) ABSTRACT

An antibacterial spray release apparatus for releasing sanitizer into the air includes a housing having a fan aperture extending through to a housing inside and a removable access panel. A sanitizer reservoir is coupled within the housing to store a liquid sanitizer. A pump is coupled to the sanitizer reservoir and a fan is coupled to the pump. The fan has a hub with a spray nozzle extending through to be in fluid communication with the pump. A motor is coupled within the housing inside and is in operational communication with the fan. A plurality of controls is coupled to the housing and is in operational communication with a microprocessor to run the motor and the pump. A power supply is coupled to the housing and is in operational communication with the microprocessor.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

(1) Field of the Invention.

The disclosure relates to sanitizing devices and more particularly pertains to a new sanitizing device for releasing sanitizer into the air. The present invention includes a fan to distribute sanitizer into the air in a room. The fan may be activated by an auditory cue such as a cough or sneeze.

(2) Description of Related Art including information disclosed under 37 CFR 1.97 and 1.98.

The prior art relates to sanitizing devices. Known devices typically rely on a pressurized container to release sanitizer into the air as an aerosol. Such devices may be manually or motion activated but lack a microphone to detect auditory cues. These devices are also typically not wall-mountable.

### BRIEF SUMMARY OF THE INVENTION

An embodiment of the disclosure meets the needs presented above by generally comprising a housing having a housing front side, a housing back side, a housing top side, a housing bottom side, a housing left side, and a housing right side defining a housing inside. The housing front side has a fan aperture extending through to the housing inside. The housing has a removable access panel. A sanitizer reservoir is coupled within the housing. The sanitizer reservoir stores a liquid sanitizer. The sanitizer reservoir is selectively removable and refillable through the removable access panel. A pump is coupled to the sanitizer reservoir. The pump is in fluid communication with the liquid sanitizer. A fan is coupled to the pump. The fan has a hub and a plurality of blades extending from the hub. The hub has a spray nozzle extending through to be in fluid communication with the pump. A motor is coupled within the housing inside and is in operational communication with the fan. A microprocessor is coupled within the housing inside and is in operational communication with the pump and the motor. A plurality of controls is coupled to the housing and is in operational communication with the microprocessor. A power supply is coupled to the housing and is in operational communication with the microprocessor.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of an antibacterial spray release apparatus according to an embodiment of the disclosure.

FIG. 2 is a front elevation view of an embodiment of the disclosure.

FIG. 3 is a rear elevation view of an embodiment of the disclosure.

FIG. 4 is a side elevation view of an embodiment of the disclosure.

FIG. 5 is a side elevation view of an embodiment of the disclosure.

FIG. 6 is a cross-sectional view of an embodiment of the disclosure along the line 6-6 of FIG. 2.

FIG. 7 is an isometric view of an embodiment of the disclosure.

### DETAILED DESCRIPTION OF THE INVENTION

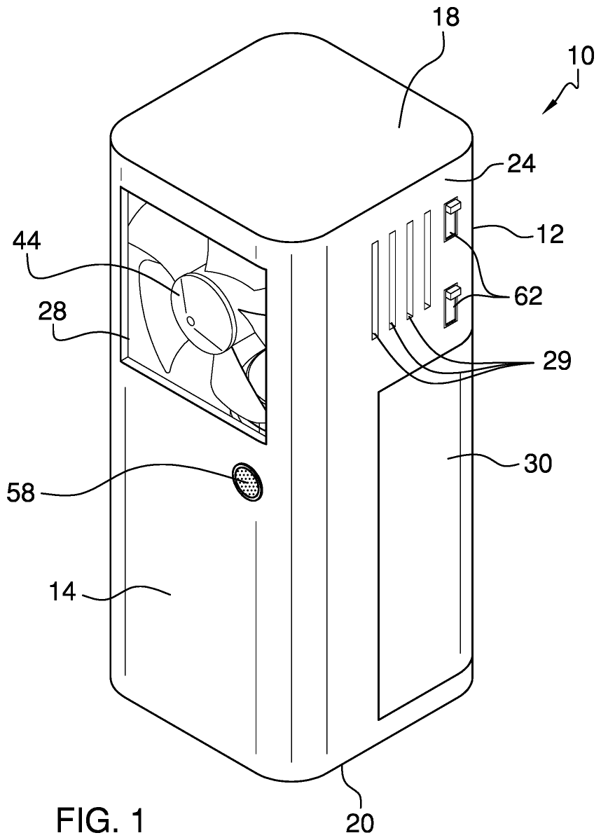

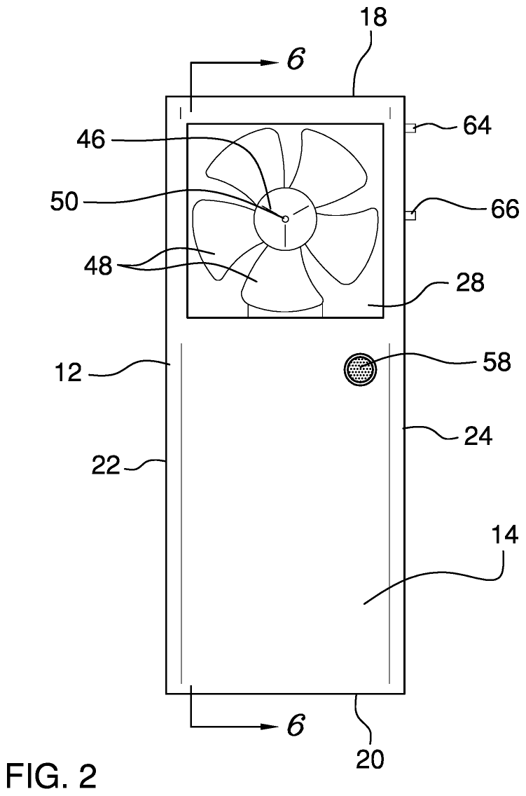

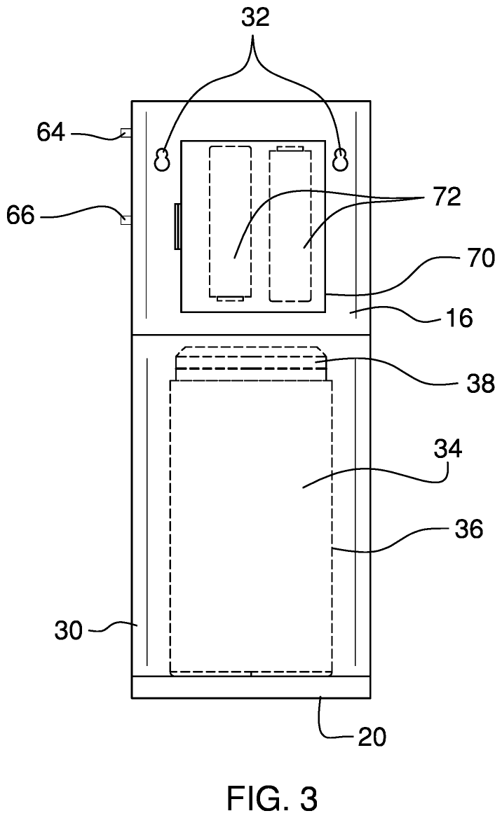

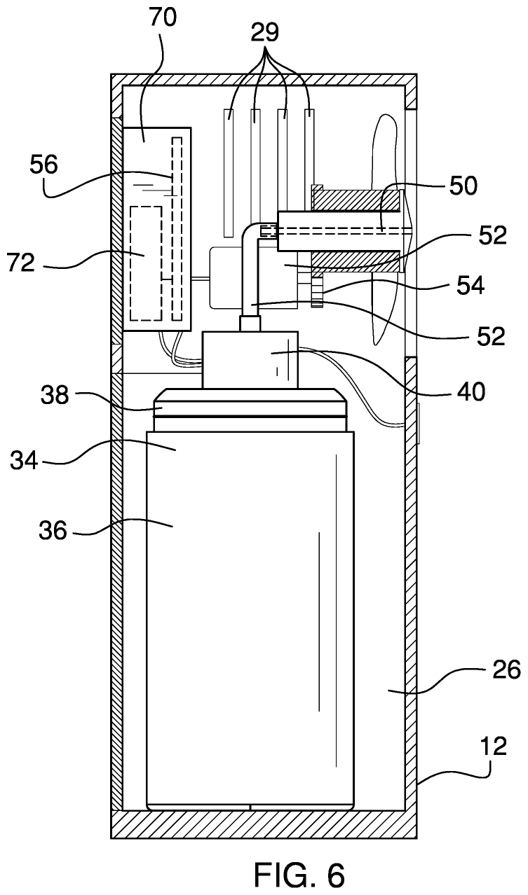

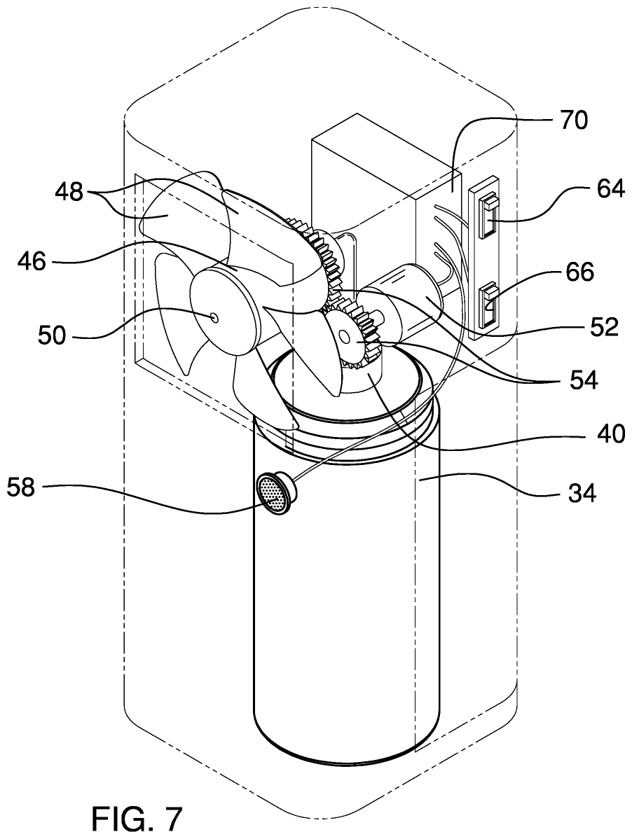

With reference now to the drawings, and in particular to FIGS. 1 through 7 thereof, a new sanitizing device embodying the principles and concepts of an embodiment of the disclosure and generally designated by the reference numeral 10 will be described.

As best illustrated in FIGS. 1 through 7, the antibacterial spray release apparatus 10 generally comprises a housing 12 having a housing front side 14, a housing back side 16, a housing top side 18, a housing bottom side 20, a housing left side 22, and a housing right side 24 defining a housing inside 26. The housing 12 may be rectangular prismatic with rounded vertical edges. The housing front side 14 has a fan aperture 28 extending through to the housing inside 26. The fan aperture 28 may be square to maximize the area. The housing right side 24 may have a plurality of intake slots 29.

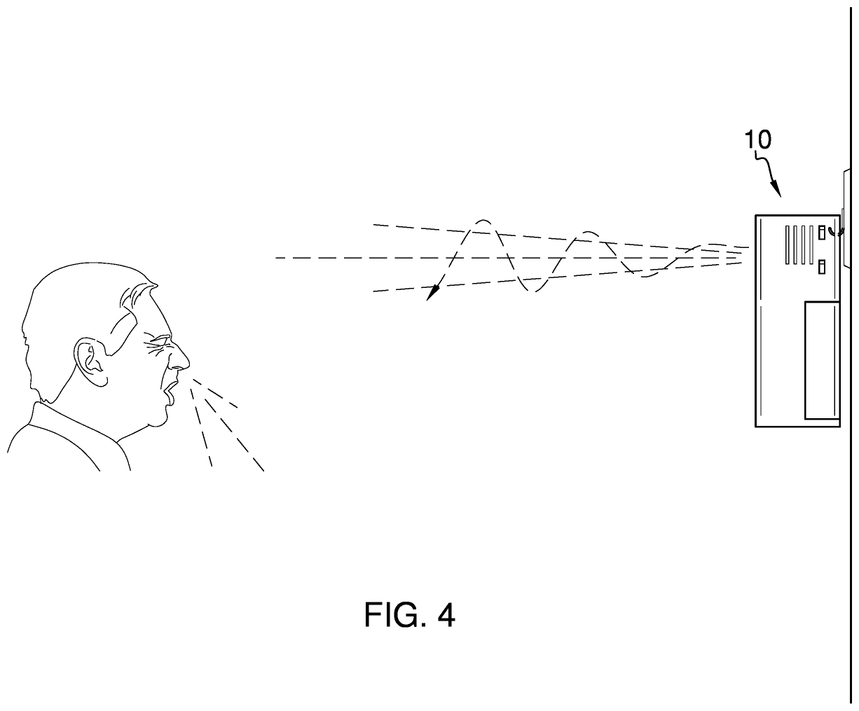

The housing 12 has a removable access panel 30 to provide access to the housing inside 26. The access panel 30 may extend from the housing back side 16 to the housing right side 24 proximal the housing bottom side 20. The housing back side 16 may have a pair of mounting apertures 32 extending through to the housing inside 26. The pair of mounting apertures 32 is configured to receive hardware to mount the apparatus to a wall as shown in FIG. 4.

A sanitizer reservoir 34 is coupled within the housing inside 26. The sanitizer reservoir 34 stores a liquid sanitizer and may include a reservoir body 36 and a reservoir cap 38. The sanitizer reservoir 34 is selectively removable and refillable through the access panel 30. A pump 40 is coupled to the sanitizer reservoir 34. The pump 40 is in fluid communication with the liquid sanitizer within the sanitizer reservoir 34 to be drawn through a connection tube 42.

A fan 44 is coupled to the pump 40. The fan 44 has a hub 46 and a plurality of blades 48 extending from the hub 46. The hub 46 has a spray nozzle 50 extending through to be in fluid communication with the connection tube 42 of the pump. The fan 44 is positioned in front of the fan aperture 28 to propel the sanitizer that has been released from the spray nozzle 50. A motor 52 is coupled within the housing inside 26. The motor 52 has a gear drive 54 in operational communication with the fan 44.

A microprocessor 56 is coupled within the housing inside 26. The microprocessor 56 is in operational communication with the pump 40 and the motor 52. A microphone 58 may be coupled to the housing 12. The microphone 58 is in operational communication with the microprocessor 56 to detect a cough or sneeze. The microphone 58 may be coupled to the housing front side 14 to provide the widest coverage should the housing 12 be wall-mounted.

A timer 60 may be coupled within the housing 12. The timer 60 is in operational communication with the microprocessor 56 to run the pump 40 and the fan 44 for a preset length of time. A plurality of controls 62 is coupled to the housing 12 and is in operational communication with the microprocessor 56. The plurality of controls 62 may include a fan setting control 64 and a timer setting control 66 coupled to the housing right side 24. The fan setting control 64 manipulates the motor 52 to create a variable fan speed.

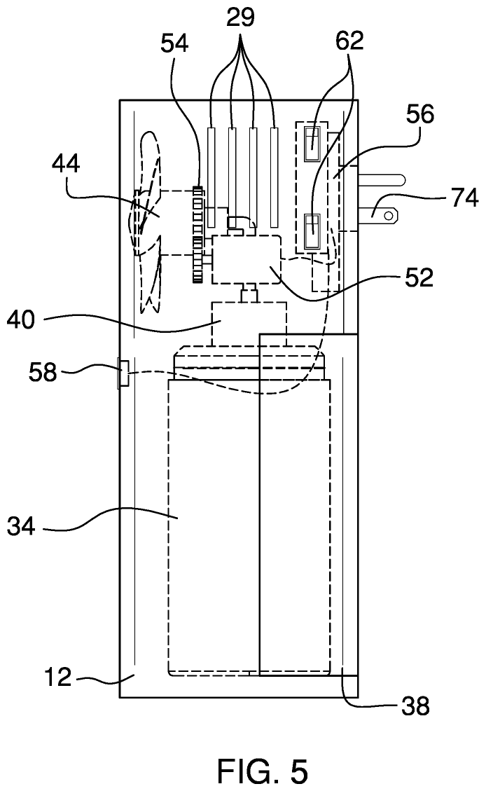

A power supply 68 is coupled to the housing 12 and is in operational communication with the microprocessor 56. The power supply 68 may include a battery compartment 70 to receive a plurality of batteries 72. The power supply 68 may include a wall plug 74 as shown in FIG. 5 to plug the apparatus 10 directly into an outlet.

In use, the plurality of controls 62 are manipulated to set the desired mode. The pump 40 and the fan 44 may run only when a sneeze or a cough is detected by the microphone 58. Alternatively, the pump 40 and the fan 44 may be activated manually and run for a preset length of time using the timer 60. When necessary, the access panel 30 is removed to refill the sanitizer reservoir 34.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An antibacterial spray release apparatus comprising:
a housing having a housing front side, a housing back side, a housing top side, a housing bottom side, a housing left side, and a housing right side defining a housing inside, the housing front side having a fan aperture extending through to the housing inside, the housing having a removable access panel;
a sanitizer reservoir coupled within the housing, the sanitizer reservoir storing a liquid sanitizer, the sanitizer reservoir being selectively removable and refillable through the removable access panel;
a pump coupled to the sanitizer reservoir, the pump being in fluid communication with the liquid sanitizer;
a fan coupled to the pump, the fan having a hub and a plurality of blades extending from the hub, the hub having a spray nozzle extending through to be in fluid communication with the pump;
a motor coupled within the housing inside, the motor being in operational communication with the fan;
a microprocessor coupled within the housing inside, the microprocessor being in operational communication with the pump and the motor;
a plurality of controls coupled to the housing, the plurality of controls being in operational communication with the microprocessor; and
a power supply coupled to the housing, the power supply being in operational communication with the microprocessor.

2. The antibacterial spray release apparatus of claim 1 further comprising a microphone coupled to the housing, the microphone being in operational communication with the microprocessor to detect a cough or sneeze.

3. The antibacterial spray release apparatus of claim 2 further comprising the microphone being coupled to the housing front side.

4. The antibacterial spray release apparatus of claim 1 further comprising the fan aperture being square.

5. The antibacterial spray release apparatus of claim 1 further comprising the access panel extending from the housing back side to the housing right side proximal the housing bottom side.

6. The antibacterial spray release apparatus of claim 1 further comprising a timer coupled within the housing, the timer being in operational communication with the microprocessor.

7. The antibacterial spray release apparatus of claim 6 further comprising the plurality of controls including a fan setting control and a timer setting control coupled to the housing right side.

8. The antibacterial spray release apparatus of claim 1 further comprising the housing back side having a pair of mounting apertures extending through to the housing inside, the pair of mounting apertures being configured to receive hardware to mount the apparatus to a wall.

9. The antibacterial spray release apparatus of claim 1 further comprising the motor having a gear drive in operational communication with the fan.

10. The antibacterial spray release apparatus of claim 1 further comprising the power supply including a battery compartment to receive a plurality of batteries.

11. The antibacterial spray release apparatus of claim 1 further comprising the power supply including a wall plug.

12. An antibacterial spray release apparatus comprising:
a housing having a housing front side, a housing back side, a housing top side, a housing bottom side, a housing left side, and a housing right side defining a housing inside, the housing front side having a fan aperture extending through to the housing inside, the fan aperture being square, the housing having a removable access panel, the access panel extending from the housing back side to the housing right side proximal the housing bottom side, the housing back side having a pair of mounting apertures extending through to the housing inside, the pair of mounting apertures being configured to receive hardware to mount the apparatus to a wall;
a sanitizer reservoir coupled within the housing, the sanitizer reservoir storing a liquid sanitizer, the sanitizer reservoir being selectively removable and refillable through the removable access panel;
a pump coupled to the sanitizer reservoir, the pump being in fluid communication with the liquid sanitizer;
a fan coupled to the pump, the fan having a hub and a plurality of blades extending from the hub, the hub having a spray nozzle extending through to be in fluid communication with the pump;
a motor coupled within the housing inside, the motor having a gear drive in operational communication with the fan;
a microprocessor coupled within the housing inside, the microprocessor being in operational communication with the pump and the motor;
a microphone coupled to the housing, the microphone being in operational communication with the microprocessor to detect a cough or sneeze, the microphone being coupled to the housing front side;
a timer coupled within the housing, the timer being in operational communication with the microprocessor;
a plurality of controls coupled to the housing, the plurality of controls being in operational communication with the microprocessor, the plurality of controls including a fan setting control and a timer setting control coupled to the housing right side; and
a power supply coupled to the housing, the power supply being in operational communication with the microprocessor, the power supply including a battery compartment to receive a plurality of batteries.

13. An antibacterial spray release apparatus comprising:
a housing having a housing front side, a housing back side, a housing top side, a housing bottom side, a housing left side, and a housing right side defining a housing inside, the housing front side having a fan aperture extending through to the housing inside, the fan aperture being square, the housing having a removable access panel, the access panel extending from the housing back side to the housing right side proximal the housing bottom side, the housing back side having a pair of mounting apertures extending through to the housing inside, the pair of mounting apertures being configured to receive hardware to mount the apparatus to a wall;
a sanitizer reservoir coupled within the housing, the sanitizer reservoir storing a liquid sanitizer, the sanitizer reservoir being selectively removable and refillable through the removable access panel;
a pump coupled to the sanitizer reservoir, the pump being in fluid communication with the liquid sanitizer;
a fan coupled to the pump, the fan having a hub and a plurality of blades extending from the hub, the hub having a spray nozzle extending through to be in fluid communication with the pump;
a motor coupled within the housing inside, the motor having a gear drive in operational communication with the fan;
a microprocessor coupled within the housing inside, the microprocessor being in operational communication with the pump and the motor;
a microphone coupled to the housing, the microphone being in operational communication with the microprocessor to detect a cough or sneeze, the microphone being coupled to the housing front side;
a timer coupled within the housing, the timer being in operational communication with the microprocessor;
a plurality of controls coupled to the housing, the plurality of controls being in operational communication with the microprocessor, the plurality of controls including a fan setting control and a timer setting control coupled to the housing right side; and
a power supply coupled to the housing, the power supply being in operational communication with the microprocessor, the power supply including a wall plug.
